(19) United States
(12) Patent Application Publication
(10) Pub. No.: US 2023/0330431 A1
(43) Pub. Date: Oct. 19, 2023

(54) PHOTOTHERAPY

(71) Applicant: Luminance Medical Ventures Incorporated, Dallas, TX (US)

(72) Inventor: Troy A. Stites, Dallas, TX (US)

(21) Appl. No.: 17/744,591

(22) Filed: May 13, 2022

Related U.S. Application Data
(63) Continuation-in-part of application No. 17/722,260, filed on Apr. 15, 2022, now abandoned.

(51) Int. Cl. A61N 5/06

(52) U.S. Cl. CPC A61N 5/0616; A61N 2005/0642; A61N 2005/0644; A61N 2005/0651; A61N 2005/0659; A61N 2005/0663

(57) ABSTRACT

The disclosure describes a phototherapy device comprising a processor including a first monochromatic LED configured to emit a first beam of light having a first discrete wavelength. In some examples, the processor further comprises a second monochromatic LED configured to emit a second beam of light having a second discrete wavelength. According to some examples, the phototherapy device further comprises a housing that contains the processor, the housing having a distal handle end and a proximal handle end opposite the distal handle end, wherein a light emitted from an LED selected from the group consisting of the first monochromatic LED and the second monochromatic LED is configured to exit the housing from the distal handle end. The phototherapy device may comprise a first actuator mechanically coupled to the housing and electrically coupled to an LED selected from the group consisting of the first monochromatic LED and the second monochromatic LED.

### FIELD

This disclosure relates to phototherapy treatments.

### DESCRIPTION OF RELATED ART

Phototherapy has recently been recognized as a viable, effective treatment option for an array of skin conditions, such as Herpes Simplex Virus (HSV)-1-related conditions (such as cold sores or canker sores), HSV-2-related conditions (such as genital herpes), and acne. Spot treatment therapy is an effective means for an individual to treat small regions that have a skin condition, without having to expose the rest of their body to a phototherapeutic device. Additionally, spot treatment therapy, because it does not cover a wide range of area, can be effectively miniaturized to the point of being placed into a handheld device, allowing for users to take their phototherapy with them, as well as more easily control the direction at which the light is pointing.

### SUMMARY

The disclosure describes a phototherapy device comprising a processor including a first monochromatic LED configured to emit a first beam of light having a first discrete wavelength. In some examples, the processor further comprises a second monochromatic LED configured to emit a second beam of light having a second discrete wavelength. According to some examples, the phototherapy device further comprises a housing that contains the processor, the housing having a proximal handle end and a distal handle end opposite the proximal handle end, wherein a light emitted from an LED selected from the group consisting of the first monochromatic LED and the second monochromatic LED is configured to exit the housing from the distal handle end. The phototherapy device may comprise a first actuator mechanically coupled to the housing and electrically coupled to an LED selected from the group consisting of the first monochromatic LED and the second monochromatic LED.

In some examples, the phototherapy device further comprises an optical extender configured to direct the first beam of light or the second beam of light, the optical extender coupled to a distal handle end of the housing. The optical extender may be removably coupled to the housing. In some examples, the optical extender is rotatably coupled to the housing. According to some examples, the optical extender further comprises at least one O-ring.

According to some examples, the phototherapy device further comprises a cover removably coupled to the optical extender. The optical extender may define a bend configured to redirect the first beam of light or the second beam of light.

In some examples, the phototherapy device further comprises a second actuator. According to some examples, the first actuator is electrically coupled to the first monochromatic LED and the second actuator may be electrically coupled to the second monochromatic LED.

In some examples, the phototherapy device further comprises a third actuator configured to cyclically actuate the first monochromatic LED and the second monochromatic LED according to a predetermined frequency.

According to some examples, the phototherapy device is configured to cause the first monochromatic LED to provide light for a first amount of time before the phototherapy device cycles to the second monochromatic LED. The phototherapy device is configured to cause the second monochromatic LED to provide light for a second amount of time before the phototherapy device cycles the second monochromatic LED to an off state. In some examples, the first amount of time is between 55 seconds and 65 seconds and the second amount of time is between 55 seconds and 65 seconds. According to some examples, the first amount of time is between 85 seconds and 95 seconds and the second amount of time is between 85 seconds and 95 seconds.

The first discrete wavelength of light may be in a visible red spectrum between 640 nanometers and 680 nanometers. In some examples, the first discrete wavelength of light is in a visible red spectrum between 610 nanometers and 650 nanometers.

According to some examples, the first discrete wavelength of light is in a visible blue spectrum between 395 nanometers and 435 nanometers. The second discrete wavelength of light may be in a visible red spectrum between 640 nanometers and 680 nanometers.

In some examples, the first discrete wavelength of light is in an invisible near-infrared spectrum between 1052 nanometers and 1092 nanometers. According to some examples, the second discrete wavelength of light is in a visible red spectrum between 640 nanometers and 680 nanometers. The phototherapy device may be configured to emit light from the first monochromatic LED and the second monochromatic LED at the same time.

This disclosure also describes example techniques for treating skin conditions through the use of a phototherapy device. In some examples, the phototherapy device includes a processor having a first monochromatic light-emitting diode (LED) configured to emit a first beam of light having a first discrete wavelength. According to some examples, the processor includes a second monochromatic LED configured to emit a second beam of light having a second discrete wavelength. The phototherapy device may include a housing that contains the processor, the housing having a distal handle end and a proximal handle end opposite the distal handle end, wherein an LED selected from the group consisting of the first monochromatic LED and the second monochromatic LED is configured to emit light through the housing at the distal handle end. In some examples, the phototherapy device includes a first actuator mechanically coupled to the housing and electrically coupled to the first monochromatic LED or the second monochromatic LED. According to some examples, the technique for treating a skin condition further includes activating, via the first actuator, an LED selected from the group consisting of the first monochromatic LED and the second monochromatic LED. The technique for treating a skin condition further includes directing, via the distal handle end of the housing, a light emitted from an LED selected from the group consisting of the first monochromatic LED and the second monochromatic LED onto a region of a user's skin.

In some examples, the phototherapy device further includes an optical extender configured to direct a beam of light selected from the group consisting of the first beam of light and the second beam of light, the optical extender coupled to a distal handle end of the housing. In some such examples, the technique for treating a skin condition further includes directing, via the optical extender, a light emitted from an LED selected from the group consisting of the first monochromatic LED and the second monochromatic LED onto the region of the user's skin.

According to some examples, the optical extender includes a first portion and a second portion angularly disposed with respect to one another and defining an extender bend, whereby the first and/or second beams of light may be preferentially redirected relative to an axis running perpendicular to the distal end of the housing. In some such examples, the technique for treating a skin condition may further include rotating the optical extender within the distal handle end of the housing, the rotation configured to change a direction of the discrete wavelength of light with respect to the housing.

Various skin conditions may benefit from differing treatment modalities or techniques. In some examples, a skin condition may benefit from treatment with either the first or second LED operating alone. In other examples, a skin condition may benefit from treatment with both the first and second LEDs operating in series or simultaneously. Treatment times may vary according to the condition being treated and the LED being utilized in the treatment.

In some examples, the technique for treating a skin condition further includes activating the first monochromatic LED. According to some examples without an optical extender, the method of treating a skin condition further includes holding the distal handle end of the housing a distance away from a treatment site for a first amount of time. Alternatively, in some examples including an optical extender, the optical extender defining a proximal extender end for coupling with the distal handle end, and a distal extender end from which the light emits, the method of treating a skin condition further includes holding the distal extender end a distance away from the treatment site for the first amount of time. The method of treating a skin condition may further comprise deactivating, after the first amount of time, the first monochromatic LED. In some examples, the distance is between 0.4 inches and 0.6 inches.

According to some examples, the skin condition is HSV-1. In such examples, the first amount of time may be between 55 seconds and 65 seconds. Additionally, the first discrete wavelength of light is between about 640 nanometers and about 680 nanometers.

According to some examples, the skin condition is HSV-2. In such examples, the first amount of time may be between 85 seconds and 95 seconds. Additionally, the first discrete wavelength of light is between 610 nanometers and 650 nanometers.

According to some examples, the skin condition is a canker sore. In such examples, the first amount of time may be between 55 seconds and 65 seconds. Additionally, the first discrete wavelength of light is between 610 nanometers and 650 nanometers.

The method of treating a skin condition may further comprise activating, after the first amount of time, the second monochromatic LED. In some examples without an optical extender, the method of treating a skin condition further comprises holding the distal handle end a predetermined distance away from the treatment site for a second amount of time. Alternatively, in some examples including an optical extender, the optical extender defining a proximal extender end for coupling with the distal handle end, and a distal extender end from which the light emits, the method of treating a skin condition further includes holding the distal extender end a distance away from the treatment site for the second amount of time. According to some examples, the method of treating a skin condition further comprises deactivating, after the second amount of time, the second monochromatic LED. In some such examples, the distance may be between 0.4 inches and 0.6 inches.

In some examples, the skin condition is acne. According to some examples, the first amount of time is between 85 seconds and 95 seconds. The first discrete wavelength of light may be between 395 nanometers and 435 nanometers. In some examples, the second amount of time is between 85 seconds and 95 seconds. According to some examples, the second discrete wavelength of light is between 640 nanometers and 680 nanometers.

The skin condition may be a condition selected from the group consisting of HSV—and canker sores. In such examples, the first amount of time is between 55 seconds and 65 seconds. Additionally, the first discrete wavelength of light is between 1052 nanometers and 1092 nanometers. The second amount of time may be between 55 seconds and 65 seconds. Also in such examples, the second discrete wavelength of light is between 640 nanometers and 680 nanometers.

According to some examples, the skin condition is HSV-2. The first amount of time may be between 85 seconds and 95 seconds. In such examples, the first discrete wavelength of light is between 1052 nanometers and 1092 nanometers. Additionally, the second amount of time is between 85 seconds and 95 seconds. The second discrete wavelength of light may be between 640 nanometers and 680 nanometers.

In some examples, the phototherapy device further comprises a second actuator. According to some examples, the first actuator is electrically coupled to the first monochromatic LED. The second actuator may be electrically coupled to the second monochromatic LED.

In some examples, the method of treating a skin condition further comprises activating, via the first actuator, the first monochromatic LED. According to some examples, the method of treating a skin condition further comprises holding the distal handle end a distance away from a treatment site for a first amount of time. The method of treating a skin condition may further comprise deactivating, after the first amount of time, the first monochromatic LED. In some examples, the method of treating a skin condition further comprises activating, via the second actuator, the second monochromatic LED. According to some examples, the method of treating a skin condition further comprises holding the distal handle end of the housing a distance away from the treatment site for a second amount of time. The method of treating a skin condition may further comprise deactivating, after the second amount of time, the second monochromatic LED. In some examples, the distance is between 0.4 inches and 0.6 inches.

According to some examples, the skin condition is acne. In such examples, the first amount of time may be between 85 seconds and 95 seconds. Additionally, the first discrete wavelength of light is between 395 nanometers and 435 nanometers. Also in such examples, the second amount of time is between 85 seconds and 95 seconds. The second discrete wavelength of light may be between 640 nanometers ad 680 nanometers.

In some examples, the skin condition is a condition selected from the group consisting of HSV-1 and canker sores. In such examples, the first amount of time is between 55 seconds and 65 seconds. The first discrete wavelength of light may be between 1052 nanometers and 1092 nanometers. Additionally, the second amount of time is between 55 seconds and 65 seconds. Also in such examples, the second discrete wavelength of light is between 640 nanometers and 680 nanometers.

The skin condition may be HSV-2. In such examples, the first amount of time is between 85 seconds and 95 seconds. Additionally, the first discrete wavelength of light is between 1052 nanometers and 1092 nanometers. The second amount of time may be between 85 seconds and 95 seconds. Also in such examples, the second discrete wavelength of light is between 640 nanometers and 680 nanometers.

### BRIEF DESCRIPTION OF THE DRAWINGS

Features, aspects, and advantages are described below with reference to the drawings, which are intended to illustrate, but not to limit, the invention. In the drawings, like reference characters denote corresponding features consistently throughout similar examples.

FIG. 1 is a profile view of an example phototherapy device.

FIG. 2A is a front view of the phototherapy device of FIG. 1.

FIG. 2B is a cross-sectional front view of a portion of the phototherapy device of FIG. 1.

FIG. 3A is a side view of the phototherapy device of FIG. 1.

FIG. 3B is a cross-sectional side view of the phototherapy device of FIG. 1.

FIG. 4 is a bottom view of the phototherapy device of FIG. 1.

FIG. 5A is a top view of an example processor for a phototherapy device.

FIG. 5B is a side view of the processor of FIG. 5A.

FIG. 6A is a side view of an example optical extender for a phototherapy device.

FIG. 6B is a front view of the optical extender of FIG. 6A.

FIG. 7 is a flowchart illustrating an example phototherapy treatment technique using one LED.

FIG. 8 is a flowchart illustrating an example timed phototherapy treatment technique using one LED.

FIG. 9 is a flowchart illustrating an example timed phototherapy treatment using a second LED that automatically turns on after the first LED treatment, such as in FIG. 8.

FIG. 10 is a flowchart illustrating a method of a timed phototherapy treatment using a first and second LED that are user-activated after the first LED treatment, such as in FIG. 8.

### DETAILED DESCRIPTION

Although specific embodiments and examples are disclosed below, the techniques of this disclosure extend beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be integrated components or separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### COMPONENT INDEX

100— Phototherapy device
102— Handle
104— Housing
106— Control interface
108— Optical extender
110— Distal handle end
112— Proximal handle end
202— Button
202a— First button
202b— Second button
202c— Third button
204— Display
302— Battery compartment
304—Rotation
402— Charging port
502—Processor
504— First monochromatic LED
506— Second monochromatic LED
602— O-ring
604— Distal extender end
606— Proximal extender end
608— Connector

Many phototherapy treatments for skin conditions in the prior art are linear devices, causing issues for the user when trying to point a light at specific areas. A linear device is shaped in a fashion similar to a pen, i.e., there is no curvature to the housing or handle that the user holds onto. Generally ergonomics have not been a consideration in the design or use of spot treatment phototherapy devices.

Another issue in the prior art is that most phototherapy devices for spot treatment do not control the spread of light coming from the device. Because the light spreads out from the source, the power of light being applied to the treatment area diminishes, and light treatment is unnecessarily provided to areas that would not meaningfully benefit from it. For the purposes of this disclosure, “areas” and “regions”, as used in describing a location of treatment, are intended to be read as synonymous.

An additional issue in the prior art is that most phototherapy treatment devices are intended for use while making contact with the user's skin, which creates an issue of hygienics. For example, if a first condition being treated is HSV-1, a user would not want to treat another area with another condition, such as acne, after touching the HSV-1-affected area. Additionally, contact between a phototherapy treatment device with sensitive or infected skin on a user may cause further irritation of the area.

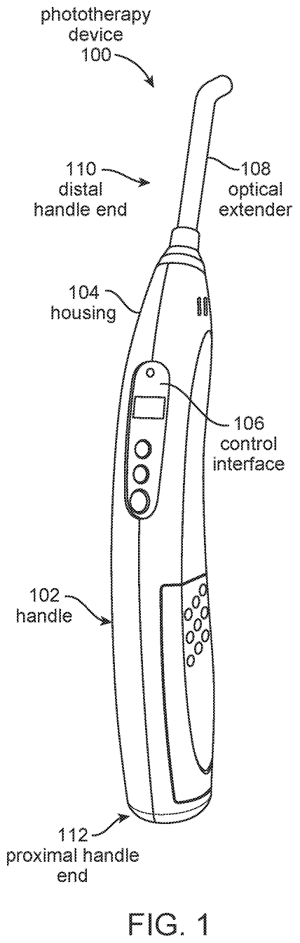

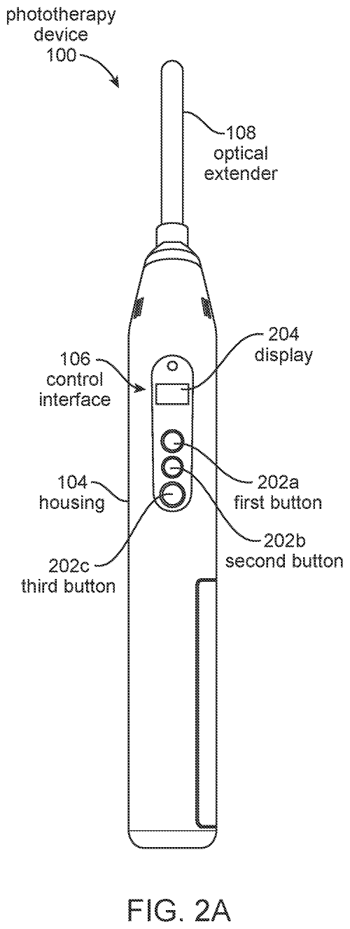

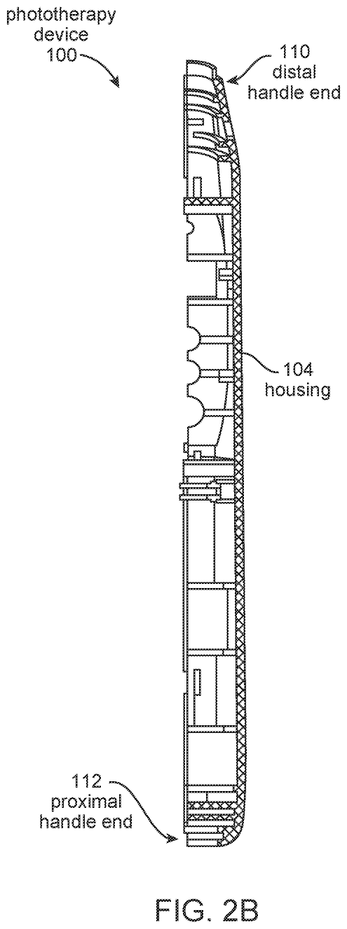

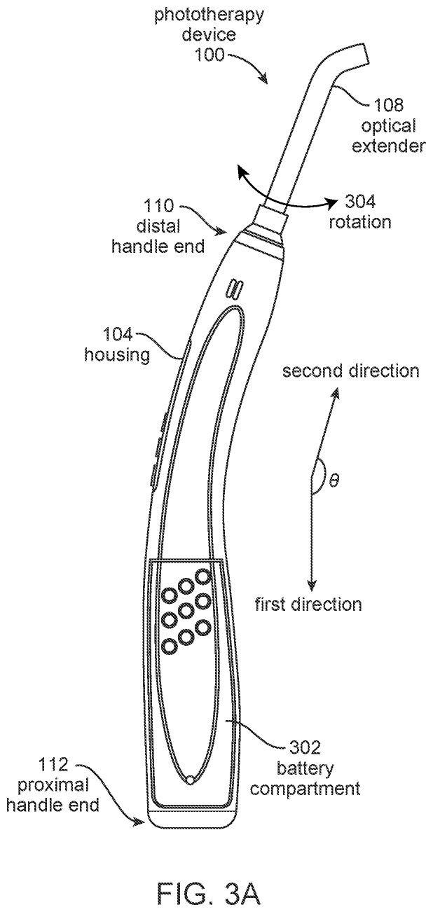

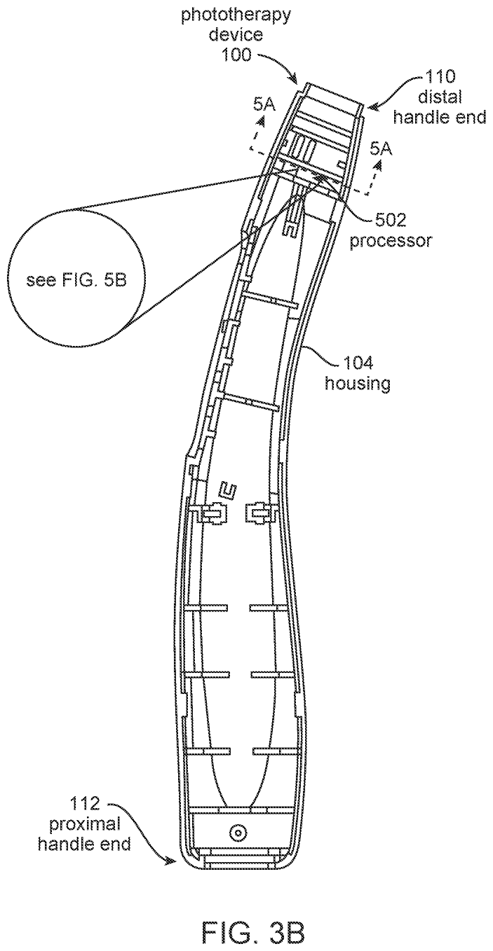

FIG. 1 is a profile view of an example phototherapy device 100, which includes a handle 102 having a housing 104 and an optical extender 108 disposed at a distal portion of the housing 104. The housing 104 at least partially contains electrical components of the phototherapy device 100, which are described in greater detail below with respect to FIGS. 2B and 3B. Throughout this disclosure, the terms “handle” and “housing” may be used interchangeably, as device 100 does not include any particular physical distinction between a manually grippable portion (e.g., a handle) and a hollow portion configured to retain one or more internal components (e.g., a housing). In other examples, however, such a distinction may be present. For instance, in other examples, the electrical components of the phototherapy device 100 need not be disposed inside of the handle 102 itself, but rather in a separately constructed housing attached to the handle 102, and handle 102 may include a solid construction.

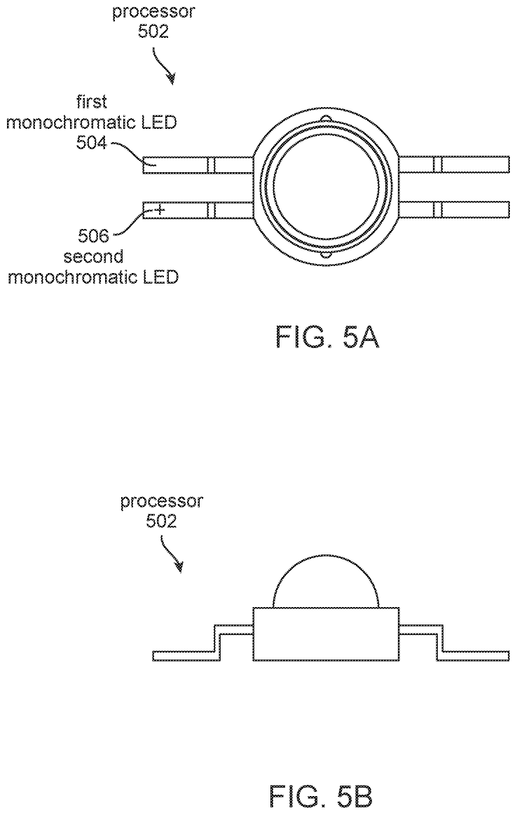

As shown in FIG. 1, the housing 104 defines a distal handle end 110 and a proximal handle end 112 positioned opposite the distal handle end 110. Phototherapy device further includes an optical extender 108 disposed at the distal handle end 110 of the housing 104. In some examples, the optical extender 108 may be removably coupled to the housing 104. The optical extender 108 is shown in greater detail in FIGS. 5A and 5B. As further shown in FIG. 1, device 100 includes a control interface 106 on the handle 102. The control interface 106 is shown in greater detail in FIG. 2A.

FIGS. 2A and 2B are front views of the phototherapy device 100 of FIG. 1. More specifically, FIG. 2A is a front view of the phototherapy device 100 including the optical extender 108, while FIG. 2B shows a cross-sectional front view of a “right” portion of the phototherapy device 100, with a “left” portion of the device removed for purposes of illustration.

FIG. 2A offers a more direct view of the control interface 106 as described above with respect to FIG. 1. The control interface 106 may include a display 204, a first button 202a, a second button 202b, and a third button 202c. The display 204 may provide information to the user, such as an indication of whether the device 100 is turned on, or an amount of time remaining in a phototherapy treatment or treatment cycle.

Throughout this disclosure, a particular one of buttons 202a, 202b, or 202c may be described as performing a particular function; however, it is to be understood that buttons 202 are fungible, and may perform any or all of the actions described herein. These actions include, but are not limited to, turning the phototherapy device 100 on and off, powering on a first monochromatic light-emitting diode (LED) 504 (FIG. 5A), powering on a second monochromatic LED 506 (FIG. 5A), or powering on both the first monochromatic LED and the second monochromatic LED 506 at the same time. According to some examples, the buttons 202 that correlate to the first monochromatic LED 504 and the second monochromatic LED 506 may actuate the LEDs for a predetermined amount of time before device 100 automatically turns the LEDs off.

Additionally, while the term “button” is used, it is understood that any type of actuator may be present to perform the described actions. For instance, a membrane switch, or a sliding mechanism or any other known actuation mechanism may be present in place of any or all of the buttons.

In some examples, where a first button 202a powers on a first monochromatic LED 504, and a second button 202b powers on a second monochromatic LED 506, a third button 202c cycles between the first monochromatic LED 504 and the second monochromatic LED 506. This cycling may swap back and forth until the third button 202c is activated again. Additionally or alternatively, the third button 202c may power on the first monochromatic LED 504 for a first period of time, after which the first monochromatic LED 504 no longer receives power. After this first period of time, the second monochromatic LED 506 is powered for a second period of time, after which the second monochromatic LED 506 no longer received power. After the second monochromatic LED 506 is powered off, the phototherapy device 100 may power off. AS described previously, while the third button 202c is described as performing these functions, it is understood that any of the buttons 202 (first button 202a, second button 202b, and third button 202c) may perform or enable these actions.

According to some examples, any of the buttons 202 make an audible sound when depressed to indicate to the user that the button 202 has been activated. Any or all of the buttons 202 may create this audible noise. In examples where the buttons 202 activate the first monochromatic LED 504 or second monochromatic LED 506 for a predetermined period of time, the audible noise may occur to indicate that the time has completed. This audible noise may indicate to a user when the treatment or cycle has finished, which may be useful in scenarios where the user is unable to see the display 204.

FIG. 2B is a diagram of the handle 102 of the phototherapy device 100 without the optical extender 108 attached. Also shown is a possible position of processor 502 (FIGS. 5A and 5B) within the housing 104 near the distal handle end 110. The processor 502 can be installed anywhere within the housing 104 so long as the beams of light from first monochromatic LED 504 and the second monochromatic LED 506 are able to exit the distal handle end 110 of the housing 104 in order to be applied to the treatment site of the user.

FIGS. 3A and 3B illustrate side views of the phototherapy device 100 of FIG. 1. More specifically, FIG. 3A shows a side view of the phototherapy device 100 including the optical extender 108, while FIG. 3B shows a cross-sectional side view of the handle 102 of phototherapy device 100 without the optical extender 108 attached. As indicated by the optical extender 108 not being present in FIG. 3B, the optical extender 108 may be detachable in some examples. Additionally, as indicated by the arrow of rotation 304 in FIG. 3A, the optical extender 108 may be rotatable while coupled to the housing 104.

FIG. 3A illustrates an embodiment, as seen in FIG. 1, wherein the housing 104 extends the entire length of the handle 102. As such, the housing 104 shows a slight curvature. This curvature occurs between the proximal handle end 112 and the distal handle end 110, and is represented by angle θ. This slight curvature increases ergonomics for a user when using the device, as it allows the user easier control for aiming the device at different parts of their own body without requiring awkward hand placement.

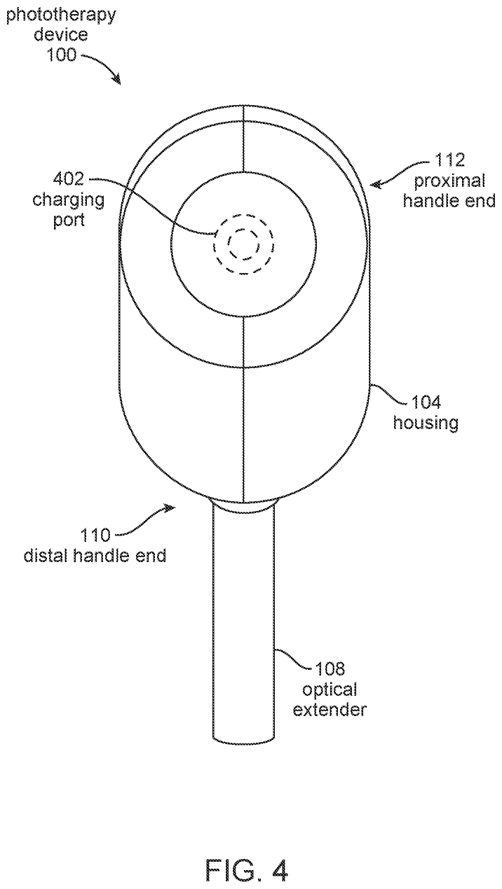

In the example shown in FIG. 3A, device 100 includes a battery compartment 302 near the proximal handle end 112 of the housing 104. The battery compartment 302 may be located anywhere on the housing 104, so long as the retained battery and associated circuitry within do not prevent the beams of light from the first monochromatic LED 504 and the second monochromatic LED 506 from exiting the distal handle end 110 of the handle 102. A standard, disposable battery may be installed in the battery compartment 302. Additionally or alternatively, the device 100 may include a rechargeable battery installed in the battery compartment 302. The phototherapy device 100 may also include a charging port 402 for charging the rechargeable battery, as described with respect to FIG. 4.

FIG. 3B illustrates an example position for the processor 502 within the housing 104. As shown, the processor 502 is slanted to allow the discrete wavelengths of light from first monochromatic LED 504 and second monochromatic LED 506 to exit the distal handle end 110 of the housing 104 at an at least partially perpendicular angle, allowing for greater control of where the light is being directed.

FIG. 4 illustrates a bottom view of the phototherapy device 100 of FIG. 1. As shown in FIG. 4, housing 104 may include a charging port 402 on the bottom (proximal handle end 112) of the housing 104. This charging port 402 may be included on phototherapy devices 100 that include rechargeable batteries. The charging port 402 is not strictly necessary and may be omitted in some examples, such as if the rechargeable battery is removed from the housing 104 for charging, or if device 100 is configured to use non-rechargeable batteries.

FIGS. 5A and 5B illustrate views of the processor 502 including first monochromatic LED 504 and second monochromatic LED 506. Specifically, FIG. 5A illustrates a top view of processor 502, and FIG. 5B illustrates a side view of the same.

As shown in FIGS. 5A and 5B, both first monochromatic LED 504 and second monochromatic LED 506 are configured to emit their discrete wavelengths of light through the same portion of the processor, which is aligned with the distal handle end of the housing, as described in FIGS. 2B and 3B. Discrete wavelengths of light as disclosed herein include wavelength ranges of 395 nanometers to 435 nanometers, 610 nanometers to 650 nanometers, 640 nanometers to 680 nanometers, and 1052 nanometers to 1092 nanometers. However, it is understood that these wavelengths of light are exemplary for applications of specific skin treatments described herein, such as HSV-1-related conditions, HSV-2-related conditions, canker sores, and acne, and any wavelengths of light may be used in conjunction with the herein described phototherapy device.

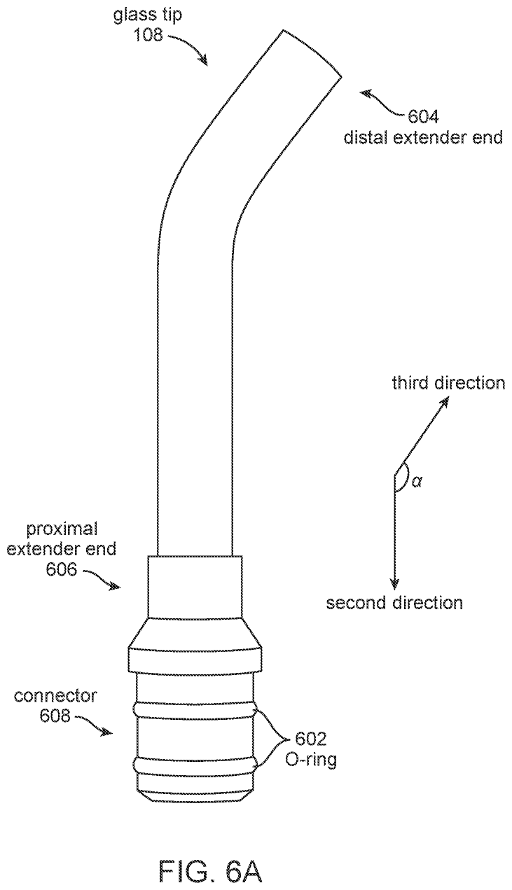

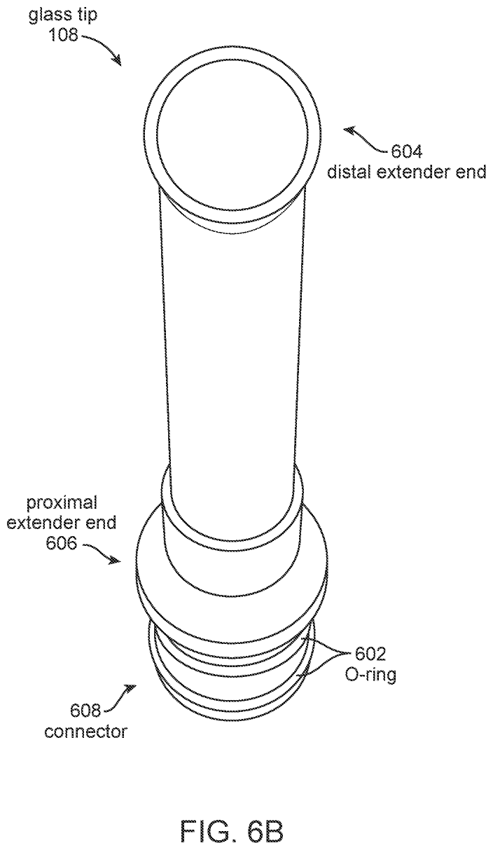

FIGS. 6A and 6B illustrate views of an optical extender 108 for a phototherapy device, such as device 100 of FIG. 1. Specifically, FIG. 6A illustrates a side view of the optical extender 108, and FIG. 6B illustrates a front view of the optical extender 108. The optical extender 108 may have a distal extender end 604 and a proximal extender end 606 opposite the distal extender end 604. According to some examples, the proximal extender end 606 includes a connector 608 for interfacing with housing 104.

FIGS. 6A and 6B show the optical extender 108 as curved, or displaying an angle (shown in FIG. 6A as angle α), so that the distal extender end 604 is not planarly parallel to the distal handle end 110 of the housing 104. Angle α is shown as existing between the second direction and a third direction. Because the proximal extender end 606 connects to the housing 104 along the same linear direction, the proximal extender end 606 is shown as extending in the second direction. The optical extender 108 may have fiberoptic capabilities, which may permit light entering the optical extender 108 to be redirected to exit the distal extender end 604. The optical extender 108 is shown as a rigid structure, but this is not strictly necessary, and flexibility may be inherent to the optical extender 108 so that a user may reposition the distal extender end 604 to a desired configuration.

As shown in FIGS. 6A and 6B, the proximal extender end 606 includes two O-rings 602 configured to permit detachable coupling of the optical extender 108 to the handle 102 of the phototherapy device 100, such that device 100 may be used with or without optical extender 108. In such embodiments, the phototherapy device 100 may be used without the optical extender 108 by holding the distal handle end 110 near the treatment site. In some examples, the O-rings 602 also enable the optical extender 108 to rotate within the housing 104 about the second direction. This rotation may further increase the ergonomics of the phototherapy device 100, by enabling the user to control a propagation direction of the light beam relative to the orientation of the user's hand placement on the handle 102.

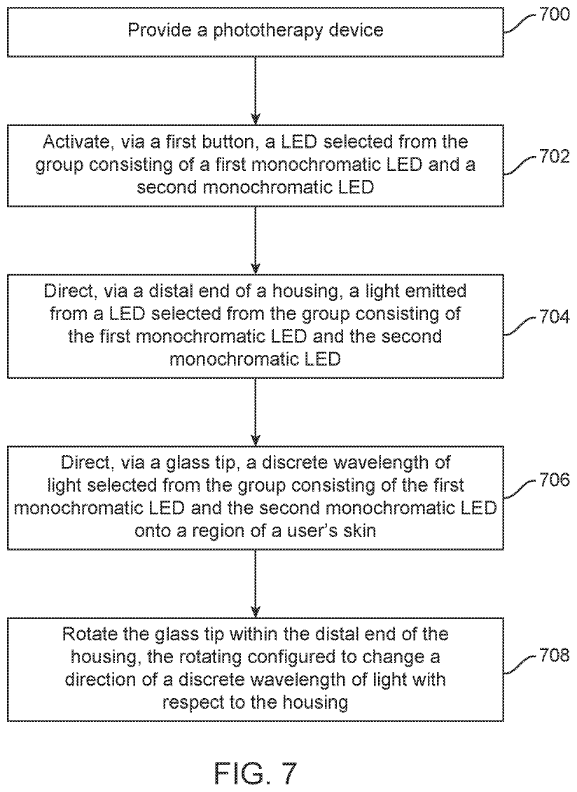

FIG. 7 is a flowchart illustrating a method of phototherapy treatment using one LED, according to some examples. In some examples, the method includes providing a phototherapy device (at step 700). According to some examples, the method includes activating, via a first button, an LED selected from the group consisting of a first monochromatic LED and a second monochromatic LED (at step 702). The method may include directing, via a distal handle end of a housing, a light emitted from an LED selected from the group consisting of the first monochromatic LED and the second monochromatic LED (at step 704). In some examples, the method includes directing, via the optical extender, a light emitted from an LED selected from the group consisting of the first monochromatic LED and the second monochromatic LED onto the region of the user's skin (at step 706). According to some examples, the method includes rotating the optical extender within the distal handle end of the housing, the rotating configured to change a direction of a beam of light with respect to the housing (at step 708).

While not shown in the flowchart of FIG. 7, the phototherapy device that is provided at step 700 may be described by any of the examples depicted in FIGS. 1-6B. That is to say, in general, the phototherapy device may comprise a first monochromatic LED capable of emitting a first discrete wavelength of light and/or a second monochromatic LED capable of emitting a second discrete wavelength of light. In some examples, the phototherapy device comprises a housing that contains the first monochromatic LED and the second monochromatic LED, the housing having a distal handle end and a proximal handle end opposite the distal handle end, wherein a light emitted from an LED selected from the group consisting of the first monochromatic LED and the second monochromatic LED is configured to exit the housing from the distal handle end. According to some examples, the phototherapy device comprises a first button mechanically coupled to the housing and electrically coupled to an LED selected from the group consisting of the first monochromatic LED and the second monochromatic LED.

Additionally, the optical extender as provided at step 706 may be responsible for directing a discrete wavelength of light selected from the group consisting of the first discrete wavelength of light and the second discrete wavelength of light. In some examples, the optical extender is coupled to a distal handle end of the housing. According to some examples, the optical extender includes a bend to change an angle of the discrete wavelength of light with respect to the housing. The optical extender may be rotatably coupled, detachably coupled, or both to the distal handle end of the housing.

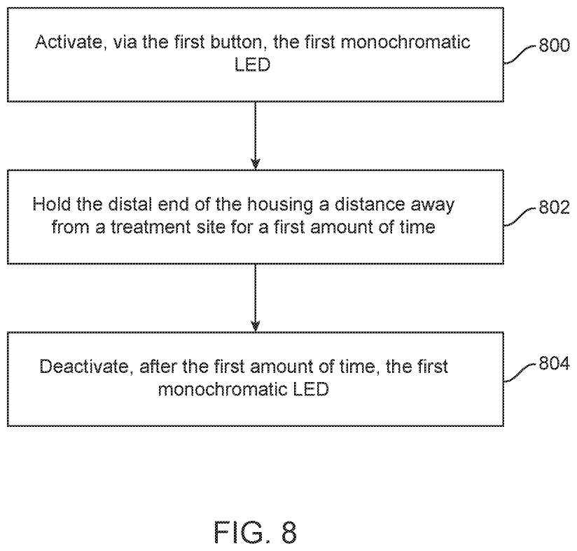

FIG. 8 is a flowchart depicting a method of a timed phototherapy treatment using one LED, according to some examples. The method of FIG. 8 is described with respect to the flowchart of FIG. 7, e.g., as optional additional steps. In some examples, the method includes activating, via the first button, the first monochromatic LED (at step 800). According to some examples, the method includes holding the distal handle end of the housing a distance away from a treatment site for a first amount of time (at step 802). The method may include deactivating, after the first amount of time, the first monochromatic LED (at step 804).

While not shown in the flowchart of FIG. 8, treatments of multiple types of skin conditions are enabled in this embodiment. For example, if the skin condition being treated is HSV-1 related, such as cold sores, the distal handle end of the housing may be held between 0.4 inches and 0.6 inches away from the treatment site for between 55 seconds and 65 seconds. In some such examples, the first discrete wavelength of light is between 640 nanometers and 680 nanometers, and perhaps exactly 660 nanometers.

If the skin condition being treated is HSV-2 related, such as genital herpes, the distal handle end of the housing may be held between 0.4 inches and 0.6 inches away from the treatment site for between 85 seconds and 95 seconds. In some such examples, the first discrete wavelength of light is between 610 nanometers and 650 nanometers, and perhaps exactly 630 nanometers.

If the skin condition being treated is a canker sore, the distal handle end of the housing may be held between 0.4 inches and 0.6 inches away from the treatment site for between 55 seconds and 65 seconds. In some such examples, the first discrete wavelength of light is between 610 nanometers and 650 nanometers, and perhaps exactly 630 nanometers.

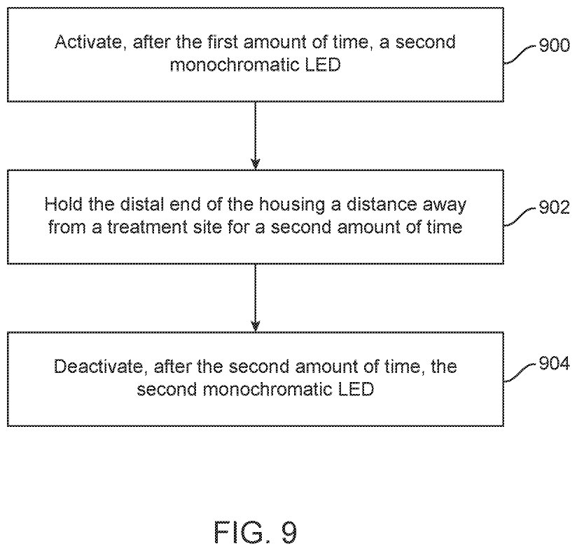

FIG. 9 is a flowchart depicting a method of a timed phototherapy treatment using phototherapy device configured to automatically actuate a second LED after a first LED treatment. The method of FIG. 9 is described with respect to the flowchart of FIG. 8, e.g., as optional additional steps. In some examples, the method includes activating, after the first amount of time, a second monochromatic LED (at step 900). According to some examples, the method includes holding the distal handle end of the housing a distance away from a treatment site for a second amount of time (at step 902). The method may include deactivating, after the second amount of time, the second monochromatic LED (at step 904).

While not shown in the flowchart of FIG. 9, treatments of multiple types of skin conditions are enabled in this embodiment. For example, if the skin condition being treated is acne, the distal handle end of the housing may be held between 0.4 inches and 0.6 inches away from the treatment site for between 85 seconds and 95 seconds. In some such examples, the first discrete wavelength of light is between 395 nanometers and 435 nanometers, and perhaps exactly 415 nanometers. After this first discrete wavelength of light has been exposed to the treatment site for between 85 seconds and 95 seconds, the second discrete wavelength of light may be exposed to the treatment site. In some such examples, the end of the housing may be held, again, between 0.4 inches and 0.6 inches away from the treatment site for between 85 seconds and 95 seconds. This second discrete wavelength of light may be between 640 nanometers and 680 nanometers, and perhaps exactly 660 nanometers.

If the skin condition being treated is HSV-1 related, such as cold sores, or canker sores, the method includes holding the distal handle end of the housing between 0.4 inches and 0.6 inches away from the treatment site for between 55 seconds and 65 seconds. In some such examples, the first discrete wavelength of light is between 1052 nanometers and 1092 nanometers, and perhaps exactly 1072 nanometers. After this first discrete wavelength of light has been exposed to the treatment site for between 55 seconds and 65 seconds, the second discrete wavelength of light may be exposed to the treatment site. In some such examples, the end of the housing may be held, again, between 0.4 inches and 0.6 inches away from the treatment site for between 55 seconds and 65 seconds. This second discrete wavelength of light may be between 640 nanometers and 680 nanometers, and perhaps exactly 660 nanometers.

If the skin condition being treated is HSV-2 related, such as genital herpes, the method includes holding the distal handle end of the housing between 0.4 inches and 0.6 inches away from the treatment site for between 85 seconds and 95 seconds. In some such examples, the first discrete wavelength of light is between 1052 nanometers and 1092 nanometers, and perhaps exactly 1072 nanometers. After this first discrete wavelength of light has been exposed to the treatment site for between 85 seconds and 95 seconds, the second discrete wavelength of light may be exposed to the treatment site. In some such examples, the method includes holding the end of the housing, again, between 0.4 inches and 0.6 inches away from the treatment site for between 85 seconds and 95 seconds. This second discrete wavelength of light may be between 640 nanometers and 680 nanometers, and perhaps exactly 660 nanometers.

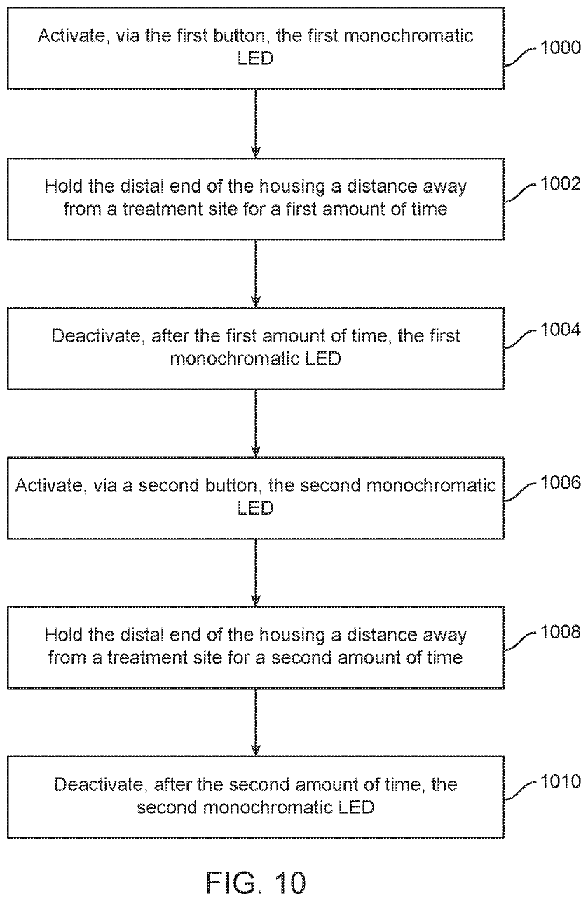

FIG. 10 is a flowchart depicting a method of a timed phototherapy treatment using phototherapy device configured to actuate a second LED in response to manual user input after a first LED treatment. The method of FIG. 10 is described with respect to the flowchart of FIG. 8, e.g., as optional additional steps. In some examples, the method includes activating, via the first button, the first monochromatic LED (at step 1000). According to some examples, the method includes holding the distal handle end of the housing a distance away from a treatment site for a first amount of time (at step 1002). The method may include deactivating, after the first amount of time, the first monochromatic LED (at step 1004). In some examples, the method includes activating, via a second button, the second monochromatic LED (at step 1006). According to some examples, the method includes holding the distal handle end of the housing a distance away from a treatment site for a second amount of time (at step 1008). The method may include deactivating, after the second amount of time, the second monochromatic LED (at step 1010).

While not shown in the flowchart of FIG. 10, the phototherapy device may include a second button in addition to the first button, as described previously in FIGS. 1-6B. In some such examples, the first button is electrically coupled to the first monochromatic LED and the second button is electrically coupled to the second monochromatic LED. The treatments for the various skin conditions described with respect to FIG. 9 (i.e., acne, HSV-1 related such as cold sores, canker sores, and HSV-2 related such as genital herpes) are much the same, with the exception that after the first amount of time has passed in each treatment, instead of the second beam of light being automatically controlled by the phototherapy device to turn on, the user is responsible for activating this second discrete wavelength of light. This may provide for additional control by the user, or different treatment times should the user desire to stop a specific treatment prematurely.

### Interpretation

None of the steps described herein are essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

1. A phototherapy device, comprising:
a housing including a proximal housing end and a distal housing end opposite the proximal housing end;
an actuator mechanically coupled to the housing;
a light emitting diode (LED) configured to emit red light in a first direction, the first direction being outward from the distal housing end, the actuator configured to activate the LED; and
an optical extender configured to redirect red light from the distal housing end, the optical extender including:
a connector located at a proximal end of the optical extender configured to detachably and communicatively couple the optical extender to the housing at the distal housing end;
an intermediate portion that extends around a perimeter of the optical extender, the intermediate portion located adjacent and distal to the connector;
a proximal extender end located distal the intermediate portion;
a tapered portion located between the intermediate portion and the proximal extender end, wherein the tapered portion tapers from the intermediate portion to the proximal extender end; and
a distal extender end located at a distal end of the optical extender;
a curved portion defining a bend between the proximal extender end and the distal extender end, the bend configured to redirect the red light with respect to the first direction,
wherein an angle of the redirected red light is between parallel and perpendicular to the first direction.

2. (canceled)

3. (canceled)

4. (canceled)

5. (canceled)

6. (canceled)

7. (canceled)

8. (canceled)

9. The phototherapy device of claim 1, wherein the LED is configured to emit light for a predetermined amount of time.

10. The phototherapy device of claim 9, wherein the LED is a first LED and the predetermined amount of time is a first predetermined amount of time, and
wherein the phototherapy device further comprises a second LED configured to emit light for a second predetermined amount of time after the first LED emits light.

11. The phototherapy device of claim 9, wherein the first predetermined amount of time is between fifty-five seconds and sixty-five seconds.

12. (canceled)

13. The phototherapy device of claim 1, wherein the LED is configured to emit a discrete wavelength of light between 640 nanometers and 680 nanometers.

14. The phototherapy device of claim 1, wherein the LED is configured to emit a discrete wavelength of light between 610 nanometers and 650 nanometers.

15. (canceled)

16. (canceled)

17. The phototherapy device of claim 10, wherein the second LED is configured to emit near infrared light between 1052 nanometers and 1092 nanometers.

18. (canceled)

19. The phototherapy device of claim 1, wherein the LED is a first LED, the phototherapy device further comprising a second LED, and
wherein the first LED and the second LED are configured to emit light at the same time.

20. (canceled)

21. The phototherapy device of claim 24, wherein the display is configured to power on in response to actuation of the actuator.

22. (canceled)

23. (canceled)

24. The phototherapy device of claim 1, further comprising a display mechanically coupled to the housing and electrically coupled to the actuator, the display configured to be accessible by a user during a treatment,
wherein the display is configured to display an amount of time remaining in a treatment.

25. The phototherapy device of claim 1,
wherein the proximal housing end defines a first diameter,
wherein the connector defines a second diameter that is less than the first diameter,
wherein the intermediate portion defines a third diameter that is less than the first diameter and greater than the second diameter,
wherein the tapered portion defines a proximal tapered end adjacent the intermediate portion and a distal tapered end adjacent the proximal extender end, wherein the tapered portion defines a fourth diameter located between the proximal tapered end and the distal tapered end, wherein the fourth diameter is less than the first diameter and the third diameter,
wherein the proximal extender end defines a fifth diameter that is less than the first diameter and the third diameter, and
wherein the curved portion defines a sixth diameter that is less than the first diameter, the third diameter, the fourth diameter, and the fifth diameter.

26. The phototherapy device of claim 25, wherein a proximal-most portion of the proximal tapered end is equal to the third diameter, and
wherein a distal-most portion of the distal tapered end is equal to the fifth diameter.

27. The phototherapy device of claim 25, wherein the tapered portion defines a linear taper beginning at the intermediate portion and tapering downward to the proximal extender end.

28. The phototherapy device of claim 25, further comprising a first O-ring coupled to the proximal extender end, wherein the first O-ring defines a seventh diameter that is less than the first diameter and greater than the second diameter.

29. The phototherapy device of claim 28, further comprising a second O-ring coupled to the proximal extender end located distal to the first O-ring.

30. The phototherapy device of claim 1, further comprising an internal housing accessibility line that extends at least partially around the housing, the internal housing accessibility line configured to allow access to an internal portion of the housing.

31. The phototherapy device of claim 30, wherein the internal housing accessibility line is configured to allow access to a battery.

32. The phototherapy device of claim 31, wherein the internal housing accessibility line is a first internal housing accessibility line, the phototherapy device further comprising a second internal housing accessibility line located between the first internal housing accessibility line and the proximal housing end, wherein the second internal housing accessibility line is configured to allow access to the internal portion of the housing.

33. The phototherapy device of claim 30, wherein the internal housing accessibility line is located proximal to the actuator.

34. The phototherapy device of claim 1, wherein the phototherapy device is configured to operate in a first configuration in which the optical extender is coupled to the housing and the red light is directed outward from a distal tip of the optical extender, and
wherein the phototherapy device is configured to operate in a second configuration in which the optical extender is detached from the housing and the red light is directed outward from the distal housing end.
